(19) United States
(12) Patent Application Publication
(10) Pub. No.: US 2022/0044765 A1
(43) Pub. Date: Feb. 10, 2022

(54) PREPROCESSING AND CONVOLUTIONAL OPERATION APPARATUS FOR CLINICAL DECISION-MAKING ARTIFICIAL INTELLIGENCE DEVELOPMENT USING HYPERCUBIC SHAPES BASED ON BIO DATA

(71) Applicants: UIF (University Industry Foundation), Yonsei University, Seoul (KR); ITMEDIC INC., Yongin (KR)

(72) Inventors: Jae Woo SONG, Seoul (KR); Ju Beam LEE, Suwon (KR)

(21) Appl. No.: 17/509,779

(22) Filed: Oct. 25, 2021

Related U.S. Application Data
(63) Continuation-in-part of application No. PCT/KR2020/013944, filed on Oct. 13, 2020.

(30) Foreign Application Priority Data
Oct. 18, 2019 (KR) 10-2019-0129523

(51) Int. Cl. G16B 40/00; G16B 5/20; G01N 15/14; G01N 33/49

(52) U.S. Cl. CPC G16B 40/00; G16B 5/20; G01N 2015/1006; G01N 33/49; G01N 15/14

(57) ABSTRACT

The present exemplary embodiments provide a data processing device and method which apply a neural network model to hypercubic data by converting a plurality of dimensions of initial data into a table type data structure and calculating between data matching the table and a designed filter.

### CROSS-REFERENCE TO RELATED APPLICATIONS

This application is a by-pass continuation-in-part application, filed under 35 USC § 111, of International Patent Application No. PCT/KR2020/013944 filed on Oct. 13, 2020, which claims the benefit of Korean Patent Application No. 10-20190129523, filed on Oct. 18, 2019, is incorporated by reference herein in its entirety.

### TECHNICAL FIELD

The technical field of the present disclosure relates to bio data preprocessing and machine learning.

### BACKGROUND ART

The contents described in this section merely provide background information on the present exemplary embodiment but do not constitute the related art.

Flow cytometry standard (FCS) data originating from medical and biological analysis equipment (e.g., flow/image cytometry, diagnostic analyzer adopting flow cytometry technology) is composed of values representing optical/electromagnetic properties of cells (or particles having physical, hydrodynamic, and optical properties similar thereto, hereinafter referred to as cells) in a medical or biological sample. The data is interpretatively analyzed and utilized as a kind of marker associated with various diseases or medical conditions.

The (flow) cytometry is an in-vitro diagnostic (IVD) and biological analysis method that measures optical/electromagnetic properties of individual cells to produce a value related to those properties or count cells showing specific properties. The values for the optical/electromagnetic measurements, in turn, represent specific properties of individual cells such as the size, the subcellular structure, and the immunophenotype (an antigen or a group of antigens a certain kind of cells typically express).

It is a common practice to convert the FCS data into dot plot images and select a group of cells of interest that appears as a cluster of dots on the plot. However, there is no known case of treating the FCS data as a single structure and extracting features of the structure. Furthermore, no known attempt has been published or reported to find the association of the structural features thus extracted to medical or biological conditions by machine learning (e.g., learning by convolutional neural network (CNN)).

### Related Art Document

### Patent Document

(Patent Document 1) Korean Patent No. 10-1857624 (May 8, 2018)

### SUMMARY

A major object of the exemplary embodiments of the present disclosure is to apply the convolutional neural network (CNN) model to FCS data with a plurality of parameters or dimensions. The initial multi-dimensional FCS data is converted into a table-type data structure. The table thus converted represents a hypercubic space containing a formed structure that is the group of data points, each corresponding to a single cell analyzed. The exemplary embodiments present the calculation conducted on the table and a designed convolution filter to carry out convolution through the hypercubic space.

Other and further objects of the present invention which are not specifically described can be further considered within the scope and easily deduced from the following detailed description and the effect.

According to an aspect of the present embodiment, a data processing method includes preprocessing initial FCS data with table-based conversion data; and applying a filter of a neural network model to the table-based conversion data.

According to another aspect of the present embodiment, a data processing device includes a processor which is configured to preprocess initial data with table-based conversion data and apply a filter of a neural network model to the table-based conversion data.

According to still another aspect of the present embodiment, a disease diagnosis method which is performed by a computing device including one or more processors and a memory which stores one or more programs executed by the processor is provided. The computing device performs a data acquiring step of FCS data from medical/biological specimens (e.g., blood, body fluid, bone marrow, cell suspension in culture media, etc.), of a diagnosis target, a preprocessing step of transforming the initial data generated based on a plurality of parameters into coordinate values for a plurality of channels and reconfiguring the transformed data as learning data, a data learning step of extracting features from the reconfigured learning data and classifying the features to perform learning; and a disease diagnosis step of diagnosing a specific disease using the trained feature.

As described above, according to the exemplary embodiments of the present disclosure, it is possible to apply a neural network model to hypercubic data by converting a plurality of dimensions of initial data into a table type data structure and calculating between data matching the table and a designed filter.

Even if the effects are not explicitly mentioned here, the effects described in the following specification which are expected by the technical features of the present disclosure and their potential effects are handled as described in the specification of the present disclosure.

### BRIEF DESCRIPTION OF THE DRAWINGS

FIG. 1 is a view illustrating a data processing device according to an exemplary embodiment of the present disclosure;

FIG. 2 is a view illustrating table based conversion data output from a data processing device according to an exemplary embodiment of the present disclosure;

FIG. 3 is a view illustrating two-dimensional data and a two-dimensional filter processible by a data processing device according to an exemplary embodiment of the present disclosure;

FIG. 4 is a view illustrating table type conversion data for two-dimensional data processed by a data processing device according to an exemplary embodiment of the present disclosure;

FIG. 5 is a view illustrating an operation between two-dimensional data and a two-dimensional filter processible by a data processing device according to an exemplary embodiment of the present disclosure;

FIG. 6 is a view illustrating an operation of performing calculation based on table type conversion data for two-dimensional data by a data processing device according to an exemplary embodiment of the present disclosure;

FIG. 7 is a view illustrating three-dimensional data and a three-dimensional filter processible by a data processing device according to an exemplary embodiment of the present disclosure;

FIG. 8 is a view illustrating table type conversion data for three-dimensional data processed by a data processing device according to an exemplary embodiment of the present disclosure;

FIG. 9 is a view illustrating an operation between three-dimensional data and a three-dimensional filter processible by a data processing device according to an exemplary embodiment of the present disclosure;

FIG. 10 is a view illustrating an operation of performing calculation based on table type conversion data for three-dimensional data by a data processing device according to an exemplary embodiment of the present disclosure;

FIG. 11 is a view illustrating an operation of designing and disposing a filter frame according to dimension increase by a data processing device according to an exemplary embodiment of the present disclosure;

FIG. 12 is a view illustrating an operation of designing to expand a filter frame according to dimension increase by a data processing device according to an exemplary embodiment of the present disclosure;

FIG. 13 is a view illustrating an operation of expanding a fractal of a filter frame according to dimension increase by a data processing device according to an exemplary embodiment of the present disclosure;

FIG. 14 is an exemplary view illustrating an operation of downwardly shifting and skipping a row group in a cubic table as convolution filter move in a three-dimensional cubic space by a data processing device according to an exemplary embodiment of the present disclosure;

FIGS. 15 and 16 are views illustrating a data processing method according to another exemplary embodiment of the present disclosure;

FIG. 17 is an exemplary view for explaining an analysis operation of bio extraction data of the related art;

FIG. 18 is a block diagram schematically illustrating a bio extraction data based disease diagnosis device according to another exemplary embodiment of the present disclosure;

FIG. 19 is a block diagram schematically illustrating an operation configuration of a processor in a disease diagnosis device according to another exemplary embodiment of the present disclosure;

FIG. 20 is a flowchart for explaining a bio extraction data based disease diagnosis method according to another exemplary embodiment of the present disclosure;

FIG. 21 is an exemplary view for explaining an operation of diagnosing a disease using patient information and bio extraction data according to another exemplary embodiment of the present disclosure;

FIG. 22 is a block diagram for explaining an operation of diagnosing a disease using a neural network according to still another exemplary embodiment of the present disclosure;

FIG. 23 is an exemplary view for explaining an operation process of a diagnosis device in a computer according to still another exemplary embodiment of the present disclosure;

FIGS. 24 and 25 are exemplary views for explaining an operation of generating initial data based on bio extraction data according to still another exemplary embodiment of the present disclosure;

FIGS. 26 to 29 are exemplary views illustrating initial data of each of a plurality of channels according to still another exemplary embodiment of the present disclosure;

FIGS. 30 and 31 are exemplary views for explaining an operation of modifying basic data based on bio extraction data according to still another exemplary embodiment of the present disclosure; and

FIG. 32 is a view for explaining an operation of reconfiguring data based on bio extraction data according to still another exemplary embodiment of the present disclosure.

### DETAILED DESCRIPTION OF THE EMBODIMENT

Hereinafter, in the description of the present disclosure, a detailed description of the related known functions will be omitted if it is determined that the gist of the present disclosure may be unnecessarily blurred as it is obvious to those skilled in the art and some exemplary embodiments of the present disclosure will be described in detail with reference to exemplary drawings.

The present disclosure relates to a method for diagnosing a disease by preprocessing bio extraction data and a device therefor.

The present disclosure relates to a device developed as a module of diagnosis equipment to utilize raw data in a flow cytometry standard (FCS) format originating from biomedical analysis equipment for a clinical decision making using a visual recognition artificial intelligence algorithm.

According to the present disclosure, high dimensional FCS data is shaped into a hypercubic space to apply an existing visual recognition artificial algorithm. The data converted into the hypercubic shape is preprocessed to be applied to the visual recognition CNN algorithm.

The existing CNN algorithm applies a convolution filter to a two-dimensional data region having a height, a width, and a color so that it is not easy to apply the convolution filter to high-dimensional hypercubic shape data only by the existing CNN algorithm.

The present disclosure applies a hypercubic convolution filter to the entire high dimensional hypercube and combines it with a high-dimensional FCS raw data hypercubic conversion technology to be utilized as visual recognition artificial intelligence based clinical decision-making diagnosis equipment.

Flow cytometry standard (FCS) data originating from medical and biological analysis equipment is biomarker data which detects optical/electromagnetic properties of individual cells (or particles having physical, hydrodynamic, and optical properties similar thereto) in a sample and shows flow cytometry/image cell analysis results which quantitatively analyzes a number of cells and properties therefrom. The data is utilized as a marker for finding association with various disease groups.

However, there is no known case of applying machine learning to find biological/clinical meaning of each sample by comprehensively analyzing an overall morphological characteristics of FCS data.

The present disclosure provides an apparatus which converts clinical information generated during a disease and progress observing process of patients or FCS data which is analysis data generated as a biological experiment result into hypercubic data to enable image analysis machine learning and applies convolution preprocessing to the hypercubic data to enable visual recognition machine learning and find a pattern related to various diseases (for example, hematologic malignancy) or biological characteristics therefrom.

There is no known technique which converts the FCS data into a hypercube, performs the convolution on 4D or higher dimensional data, and applies CNN. Even though a medical/biological analysis FCS data conversion hypercubic shape is not considered, there is no known case of applying the convolution processing and CNN machine learning to general multivariate data corresponding to 4Dr or higher dimensional shape.

According to the present disclosure, the FCS data machine learning model development for clinical prediction is accelerated so that circumstantial and integrated interpretation of the automatic blood analysis test and flow cytometry result is possible beyond the conventional disease diagnosis method based on fragmentary numerical comparison, which may help in more accurate disease diagnosis and clinical situation identification.

According to the present disclosure, as a FCS data pattern having a clinical usefulness is discovered, medical innovation to discover abnormalities of patients which are not recognized by doctors to quickly diagnose and identify patients may be achieved. Further, automated blood analysis test which is cheaper than a disease specific test is performed to track disease progress and changes in patient status to contribute to improve the efficiency of medical resource distribution. According to the present disclosure, development of a new algorithm which automates the reading of flow cytometry test results of the related art which mainly relies on the analyst's manual work is accelerated to facilitate the biological and medical research.

The FCS data in the medical field is being produced stably and consistently in large quantities by performing the automatic blood analysis test which is a normal test. Further, regional and international quality control system of the clinical pathology which is well established may allow the mechanical performance to be maintained while achieving a very high level of standardization.

Accordingly, it is obvious that the FCS data derived from flow cytometry as well as the automatic blood analysis test is very suitable for the development of the machine learning algorithm aimed at clinical application.

The FCS data conversion which is the contents of the present disclosure has great industrial and academic values in that it may open the door of a new medical machine learning field. Moreover, it allows the machine learning to be performed on another high-dimensional data.

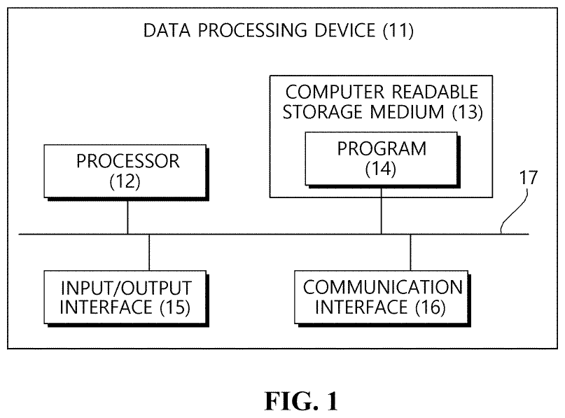

FIG. 1 is a view illustrating a data processing device according to an exemplary embodiment of the present disclosure.

The device 11 includes at least one processor 120, a computer readable storage medium 13, and a communication bus 17.

The processor 120 controls the device 11 to operate. For example, the processor 12 may execute one or more programs stored in the computer readable storage medium 130. One or more programs may include one or more computer executable instructions and the computer executable instruction may be configured to allow the device 11 to perform the operations according to the exemplary embodiments when it is executed by the processor 12.

The computer readable storage medium 13 is configured to store a computer executable instruction or program code, program data and/or other appropriate format of information. A computer executable instruction or program code, program data and/or other appropriate type of information may also be provided by an input/output interface 15 or a communication interface 16. The program 14 stored in the computer readable storage medium 13 includes a set of instructions executable by the processor 12. In one exemplary embodiment, the computer readable storage medium 13 may be a memory (a volatile memory such as a random access memory, a non-volatile memory, or an appropriate combination thereof), one or more magnetic disk storage devices, optical disk storage devices, flash memory devices, and another format of storage mediums which is accessed by the data processing device 11 and stores desired information, or an appropriate combination thereof.

The communication bus 17 includes a processor and a computer readable storage medium 13 to interconnect various components of the data processing device 11 to each other.

The device 11 may include one or more input/output interfaces 15 and one or more communication interfaces 16 which provide an interface for one or more input/output devices. The input/output interface 15 and the communication interface 16 are connected to the communication bus 17. The input/output device (not illustrated) may be connected to the other components of the device 11 by means of the input/output interface 15.

The processor 12 of the data processing device 11 preprocesses initial data into table based conversion data and applies a filter of the neural network model to the table based conversion data.

The processor 12 converts a first data structure formed with N-dimensional data by N axes (N is a natural number of 2 or larger).

The first data structure may include a hypercube having 4D or higher dimensional depth information. The first data structure may include bio-extraction data indicating a measurement result of flow cytometry of a clinical sample such as blood or a biological analysis sample and an analysis technique using flow cytometry. The bio extraction data may be expressed by a predetermined standardized format or a flow cytometry standard (FCS) format.

In the second data structure, (i) coordinate information corresponding to N axes and (ii) value information matching the coordinate information are disposed with reference to a row direction or a column direction. The second data structure merges measurement values of some parameters of the bio extraction data and transforms the measurement values into data including a coordinate value for a channel and includes the transformed data and a count value.

The processor designs a filter frame structure which is computable with a second data structure and expresses a dimension to apply a neural network model to the first data structure. The processor disposes a filter center of the filter frame structure with reference to a predetermined coordinate to set a starting position of the filter frame structure. The processor may expand filter weight elements of the filter frame structure with a fractal like pattern according to a dimension with reference to the row direction or the column direction in consideration of a dimension of the first data structure.

The processor may perform the calculation between matching elements by moving the filter frame structure with reference to the row direction or the column direction of a table of the second data structure. When the filter center of the filter frame structure satisfies a predetermined row condition or column condition, the processor may skip the calculation.

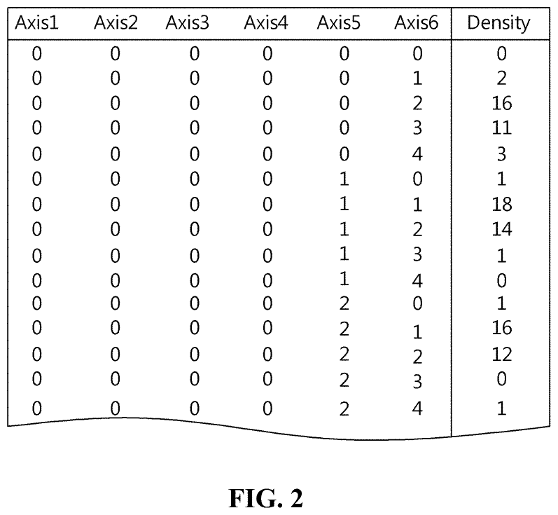

FIG. 2 is a view illustrating table based conversion data output from a data processing device according to an exemplary embodiment of the present disclosure.

According to the present exemplary embodiment, the format of a confined hypercube space is expressed as a table with two types of columns (or rows) for coordinates and gray-scale densities of hypercubic voxels. The convolution filter has the same dimension as the hypercubic space to be convoluted, but has a much smaller size.

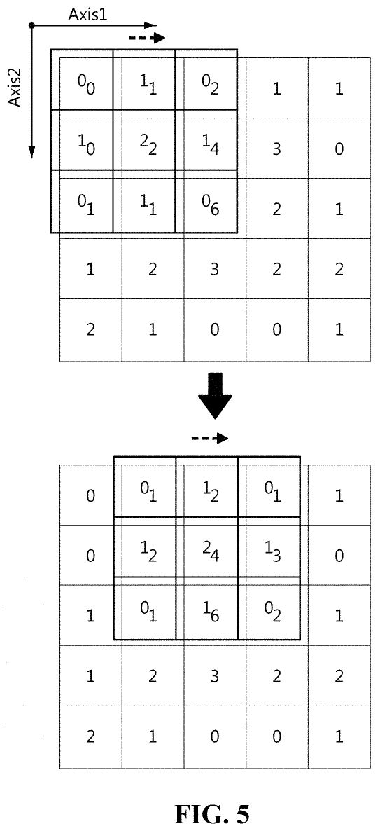

Referring to FIG. 5, a 6D hypercubic space is illustrated. Each dimension has a size of five (with an arbitrary unit for a scale). Six axes are assigned to six dimensions. A location of each voxel is represented by a coordinate. The coordinate has six components and each of which is projectional location in the corresponding dimension. Each voxel with its coordinate and gray-scale density occupies each row (or column) of this table. Even though in FIG. 2, the voxels are disposed in a row direction, the voxels may also be disposed in a column direction depending on a design. That is, row data and column data may be shifted (diagonal movement).

All voxels composing the hypercubic space are disposed in a specific order. First, in five rows at the uppermost part of the table, gray-scale densities of the voxels whose positions in a first to fifth dimensions (or coordinate values) are 0 and position in the sixth dimension is 0 to 4 ((0,0,0,0,0,0), . . . , (0,0,0,0,0,4)) are shown. In voxels of a subsequent row, a position (coordinate value) in a fifth dimension is shifted to 1 and a position (coordinate value) of a sixth dimension proceeds from 1 to 4 in the same way as the previous step. After adding rows sixth dimension positions (coordinate value) 0 to 4 to fifth dimension positions (coordinate value) 2 to 4, the whole process described above is iterated for the fourth dimension position (coordinate value) 1 to 4 ((0,0,0,1,0,0), . . . , (0,0,0,4,0,0)). The voxels are added as table rows in this manner (position shift in each dimension). A table type data structure may represent information about all 5⁶ voxels from (0,0,0,0,0,0,) to (4,4,4,4,4,4) in the hypercubic space.

The convolution assigns a weight to each voxel of the filter and multiplies a grayscale density of voxels of the hypercube overlapping the filter voxel and sums the products. Each row of the table representing a hypercubic space corresponds to a voxel so that voxels overlapping the filter may be assigned as a group of rows. When the filter passes through the hypercube in a scanning manner, the filter overlaps voxels of another hypercubic space in every movement step and the overlapping voxels form different groups of rows for every step. The group of rows corresponding to hypercubic space voxels overlapping the filter in every movement step shows a specific topological pattern or pattern in the table and moves the position together with the movement of the convolution filter. It starts from an image and a cube and then proceeds to hypercubes of a higher dimension.

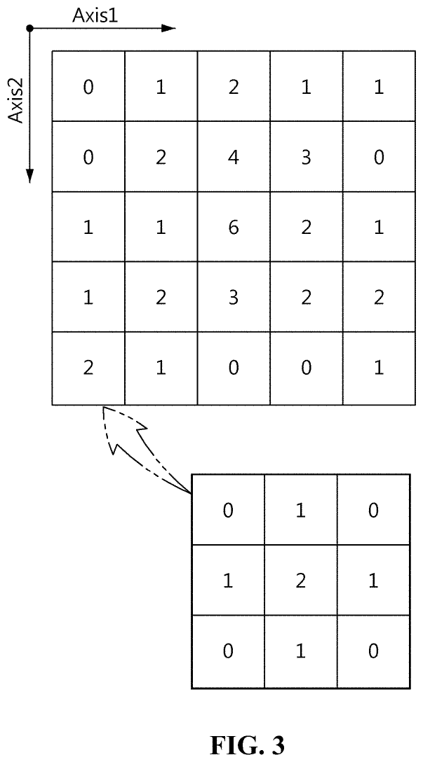

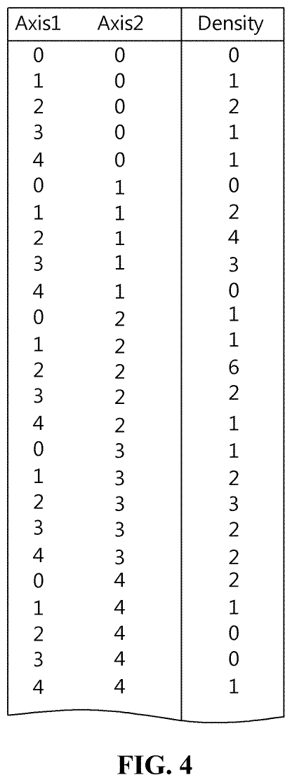

FIG. 3 is a view illustrating two-dimensional data and a two-dimensional filter processible by a data processing device according to an exemplary embodiment of the present disclosure. FIG. 4 is a view illustrating table type conversion data for two-dimensional data processed by a data processing device according to an exemplary embodiment of the present disclosure.

A first example is an image of 5×5 size. The columns labeled axis-1 and axis-2 show a projectional location (or a coordinate value) of a pixel in dimension-1 and dimension-2. The order of listing rows with location and density information is as explained above. Next, it is described to apply a 3×3 size convolution filter to an image.

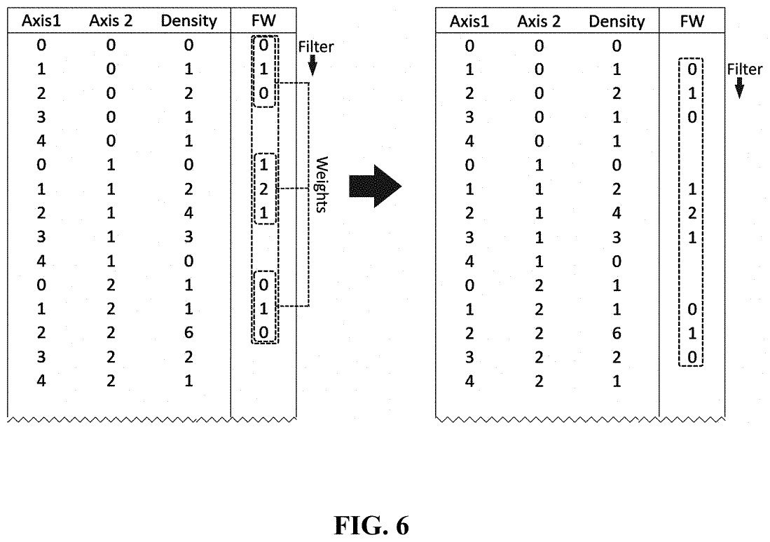

FIG. 5 is a view illustrating an operation between two-dimensional data and a two-dimensional filter processible by a data processing device according to an exemplary embodiment of the present disclosure. FIG. 6 is a view illustrating an operation of performing calculation based on table type conversion data for two-dimensional data by a data processing device according to an exemplary embodiment of the present disclosure.

The exemplary embodiment follows a rule that there is no special treatment for edges. First, the convolution is carried out by locating the filter center in a position (1,1) of the image. The filter and the image are illustrated by an overlapping matrix. Next, the filter moves to the direction of the axis-1 for a next convolution step. The convolution is to multiply the weights of the filter pixel and a density of the overlapping image pixel and sum the products.

The first convolution produces (=0×0+1×1+0×2+1×0+2×2+1×4+0×1+1×1+0×6). A second convolution is 21 (=0×1+1×2+0×1+1×2+2×4+1×3+0×1+1×6+0×2). The filter columns are added to an original table and a weight assigned to the filter pixel is represented. A row group corresponding to an image pixel overlapping a filter pixel is specified in a filter column.

In the table, rows 1, 2, 3, 6, 7, 8, 11, 12, 13 indicate image pixels overlapping the filter in the first convolution step. In the table representation of convolution, each filter weight is multiplied with the density in the same row.

A number in a filter columns labeled FW (filter weight) 1 and FW2 implies that the filter is two dimensional. In column FW2, a part of the filter may expand beyond the two dimension.

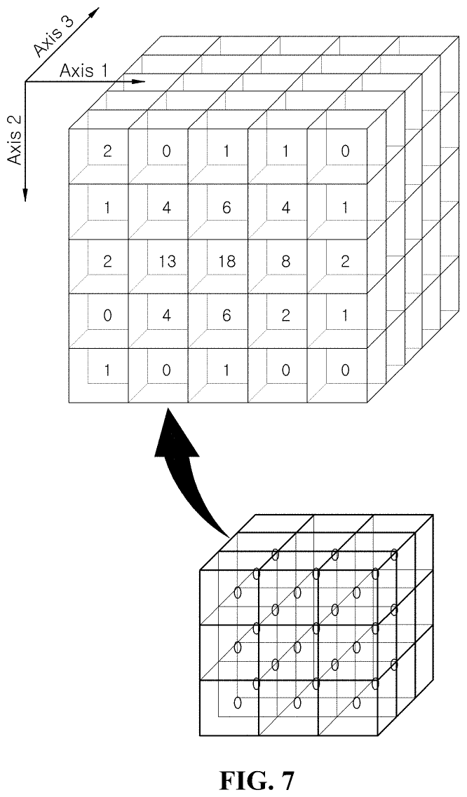

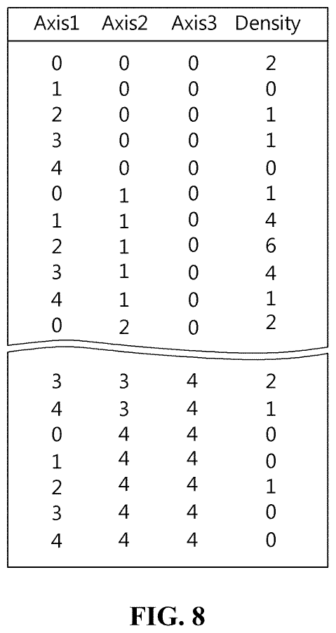

FIG. 7 is a view illustrating three-dimensional data and a three-dimensional filter processible by a data processing device according to an exemplary embodiment of the present disclosure. FIG. 8 is a view illustrating table type conversion data for three-dimensional data processed by a data processing device according to an exemplary embodiment of the present disclosure.

A second example is a cube with a 5×5×5 size. A filter with a 3×3×3 size is applied to the cube to show how it looks like in the table.

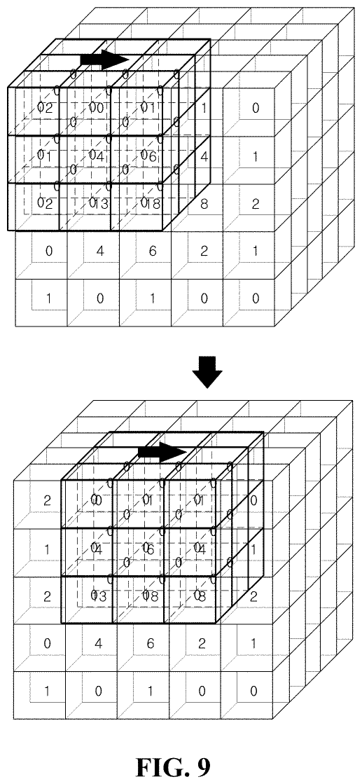

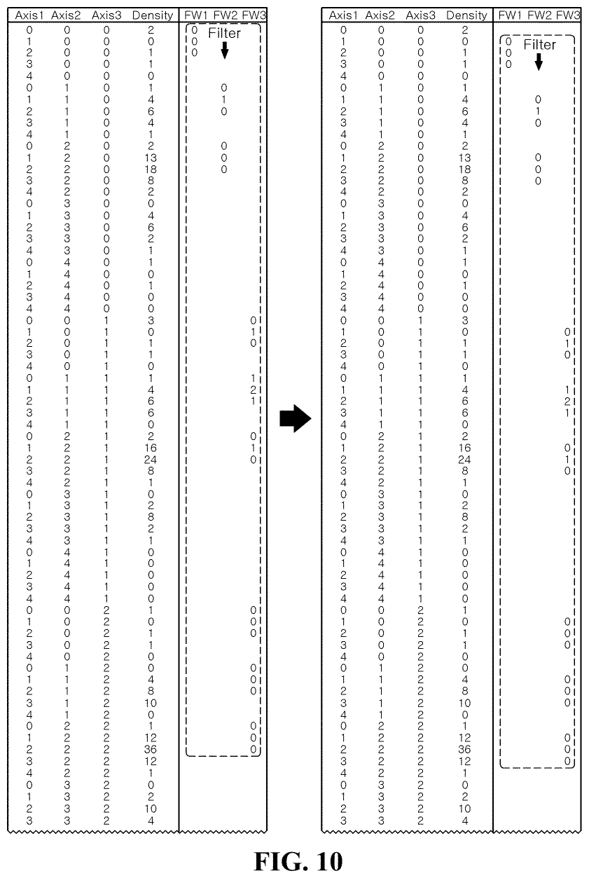

FIG. 9 is a view illustrating an operation between three-dimensional data and a three-dimensional filter processible by a data processing device according to an exemplary embodiment of the present disclosure. FIG. 10 is a view illustrating an operation of performing calculation based on table type conversion data for three-dimensional data by a data processing device according to an exemplary embodiment of the present disclosure.

The convolution is carried by locating the filter center at (1,1,1) of a cubic space. Next, the filter moves to the direction of the axis-1 for a next convolution step. Now, the filter center is at (2,1,1) in the cubic space.

A first convolution value is 39 (=0×2+0×0+0×1+0×1+1×4+0×6+0×2+0×13+0×18+0×3+1×0+0×1+1×1+2×4+1×6+0×2+1×16+0×24+0×1+0×0+0×1+0×0+1×4+0×8+0×1+0×12+0×36).

A second convolution value is 61 (=0×0+×1+0×1+0×4+1×6+0×4+0×13+0×18+0×8+0×0+1×1+0×1+1×4+2×6+1×6+0×16+1×24+0×8+0×1+0×1+0×1+0×4+1×8+0×10+0×12+0×36+0×12).

The filter columns are added to an original table and a weight assigned to the filter voxel in the column is represented. Cubic voxels (and corresponding rows) overlapping the filter voxels are specified in the filter columns. In the table, rows 1, 2, 3, 6, 7, 8, 11, 12, 13 indicate voxels of the cube overlapping the filter in the first step of convolution. In the table representation of convolution, each filter weight is multiplied with the density in the same row.

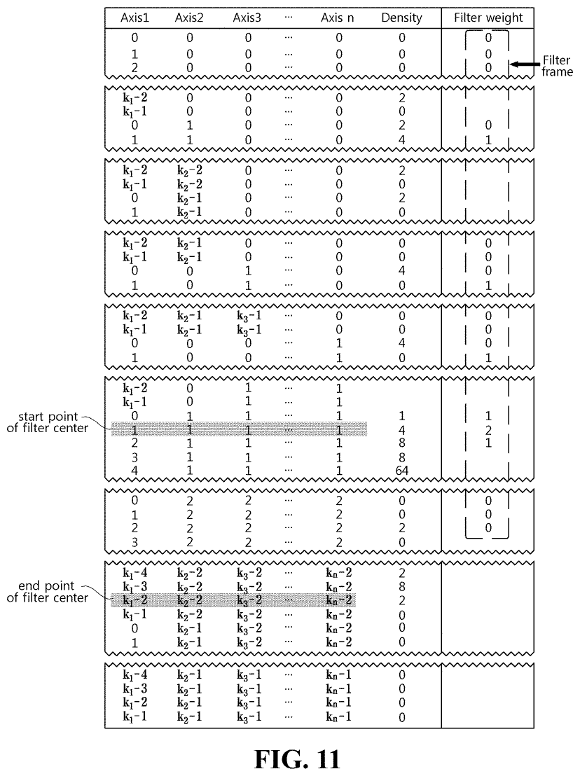

FIG. 11 is a view illustrating an operation of designing and disposing a filter frame according to dimension increase by a data processing device according to an exemplary embodiment of the present disclosure.

An example of convolution for an n-dimensional hypercubic space is presented. Referring to FIG. 11, an example of a convolution filter framework in the table (rows/columns of the filter voxels) is illustrated.

An easier way to determine a coordinate of a filter voxel within a confined hypercubic space is as follows. In this example, a number of voxels in each dimension is set to be odd in order to easily find a location of the filter center. Further, a method of using a convolution filter having the same size (represented by a number of voxels) in all dimensions will be described. For example, a hypercubic space of n-dimensions, the size of each dimension being kᵢ (i=2, . . . , n) and a hypercubic convolution filter with a size of 3ⁿ, the size of each dimension being 3 are considered.

Next, a method of selecting a coordinate of convolution filter voxels in the hypercubic space will be described. An arbitrary point (x₁, . . . , xₙ) in the hypercubic space is set as a filter center. When the coordinate of 3ⁿ filter voxels is expressed as (X₁, . . . , Xₙ), X₁ has values of x₁−1, x₁, and x₁+1, X₂ has values of x₂−1, x₂, and x₂+1 , . . . , and Xₙ [symbol] has values of xₙ−1, xₙ, and xₙ+1. The coordinate of every dimension has three values so that all 3ⁿ coordinates are possible and the locations of filter voxels and the hypercubic voxels overlapping filters are represented. When hypercubic table rows having this coordinates are chosen, it becomes a row group corresponding to the filter overlapping hypercubic voxels. When a filter center coordinate in an initial convolution step is (1, . . . , 1), a row having a coordinate (0 or 1 or 2, . . . , 0 or 1 or 2) becomes a coordinate of the remaining filter voxels. These rows are selected to determine a filter frame.

Next, a method of directly obtaining a filter overlapping row group from a row (center row) to which filter center is assigned in the hypercubic space table will be described.

First, two rows of a table adjacent to a center row are taken. Three rows selected as described above corresponds to a linear segment 3-unit long on an axis of a specific dimension (in this case, dimension-1). Next, three rows distance by k₁ above and below three rows in the previous step are added to select a total of nine rows. This task produces a 3² sized square which shares a center with the filter in the hypercube. Next, nine rows distance by k₁ x k₂ above and below nine rows in the previous step are added to select a total of 27 rows. By this operation, a 3³-sized cube with the same center as the convolution filter is produced. In the next step, 27 rows distance by k₁×k₂×k₃ above and below 27 rows are selected to produce a 3⁴-sized four-dimensional hypercube.

The same operation on the table is iterated until 3ⁿ rows corresponding to 3ⁿ-sized convolution hypercubic filter are selected.

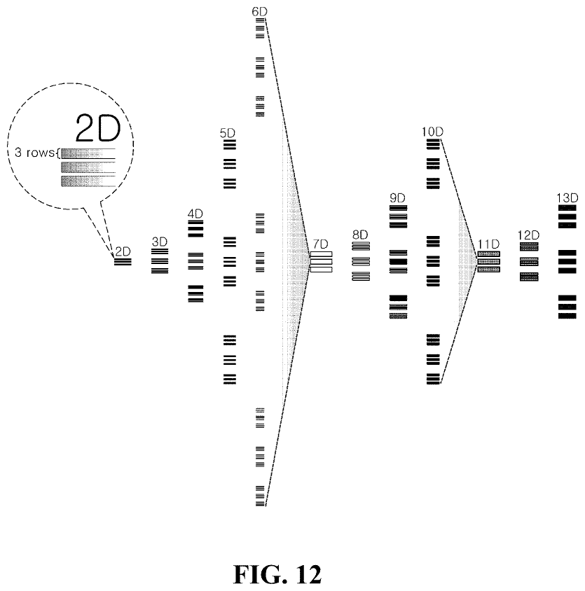

FIG. 12 is a view illustrating an operation of designing to expand a filter frame according to dimension increase by a data processing device according to an exemplary embodiment of the present disclosure.

FIG. 12 shows how the framework structure of the convolution filter expands in the table as the dimension increases. Here, the size of the convolution filter is 3 for each dimension and a topological structure of the filter (overlapping) row in the table expands with the same fractal-like pattern. The same current structure is added below and above itself as the dimension increases by one.

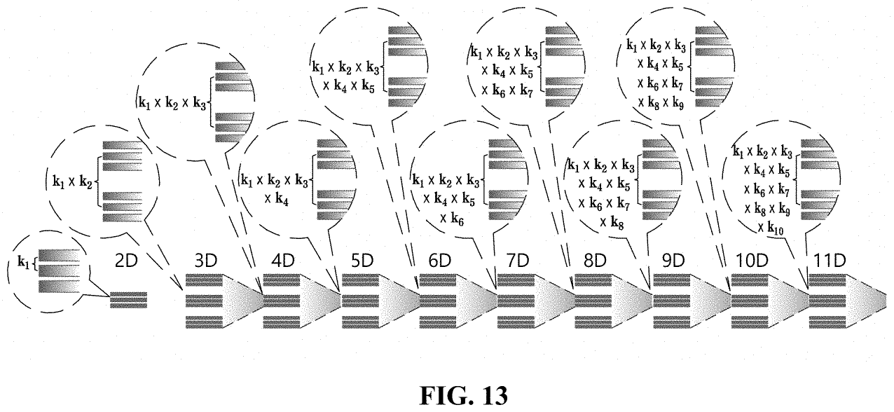

FIG. 13 is a view illustrating an operation of expanding a fractal of a filter frame according to dimension increase by a data processing device according to an exemplary embodiment of the present disclosure. In the case of the 2D filter, rows are added to the filter center, upper and lower three rows, and a location distance by k₁ above and below the three rows. In the case of the 3D, the same frame rows as the 2D filter are added with a distance of k₁×k₂ rows above and below. In the case of the 4D, the 3D filter frame is added with a distance of k₁×k₂×k₃ rows above and below.

In general terms, in the case of the n dimension, (n−1) dimensional filter frames are added above and below the (n−1) dimensional filter frames with a distance of k₁× . . . ×kₙ₋₁ rows above and below. 3ⁿ rows may be selected for the n-dimensional convolution filter by a fractal method according to dimension expansion.

The calculation for convolution is the same as the calculation for a low-dimensional space and filter and a filter voxel weight overlapping a density of gray scale of the hypercubic voxel is multiplied and the products are summed.

Hereinafter, how the filter frame of the table is changed when the filter scans the hypercubic space will be described.

First, a start point of the convolution, that is, an initial location of the filter center needs to be determined.

In the case of the 3ⁿ-sized convolution filter, a start point expressed with a coordinate is (1, . . . , 1). In the table, a row with this coordinate is a start point of the filter center.

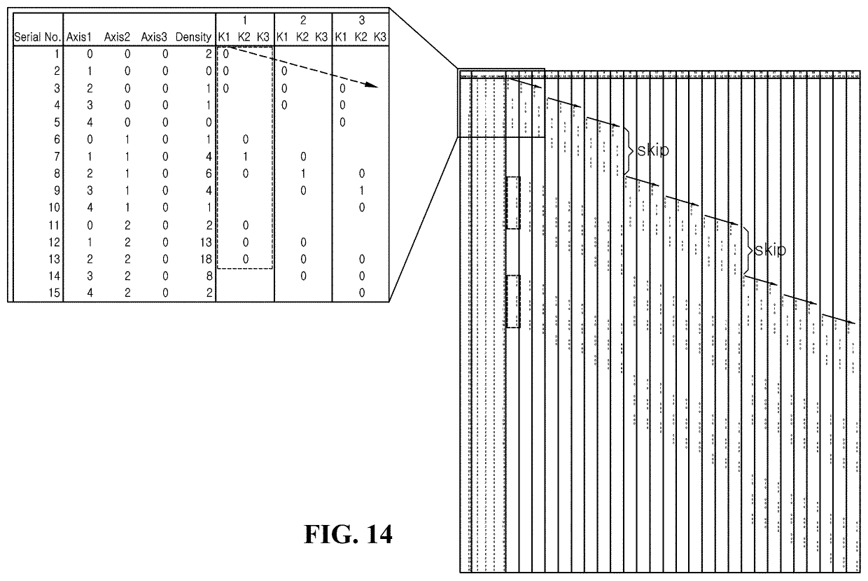

3ⁿ rows corresponding to the filter frame maintain a framework topology in the table as the filter shifts in the hypercubic space. One voxel shift of the filter in the hypercubic space is equal to one-row downward shift of the filter frame in a tabular representation. The table may be organized such that the filter frame continuously moves down the table when the hypercubic space is scanned. The table may also be organized in the other direction. When data is recorded in a column direction of the table, one voxel shift of the filter in the hypercubic space may be configured to be the same as lateral shift. FIG. 14 is an exemplary view illustrating an operation of downwardly shifting and skipping a row group in a cubic table as convolution filter move in a three-dimensional cubic space by a data processing device according to an exemplary embodiment of the present disclosure.

One line downward (or one column lateral) shift is a basic operation.

When the filter reaches the edge of a particular dimension, the following scanning movement is special. The filter shifts its position one by one in a next higher dimension. The filter returns to a starting position in the current dimension. This type of scanning movement appears as more-than-one row downward shift of the filter frame in the table. When the edge treatment is not applied, there is no convolution product when a part of the filter is beyond an end of the space. A non-productive positions of the filter should not appear in a final convolution result.

Accordingly, it is necessary to skip a certain number of filter frame positions in the table, which appears as more-than-one row downward shift of the filter frame.

The filter may continue to be at an end of the dimension despite continuous movements. For example, scanning along a surface of the space may be such a case. In this case, even more significant shift of the filter frame position is necessary.

A shifting pattern may be set with a filter frame center coordinate by a simple method. A row corresponding to a non-productive filter center position (coordinate) may be determined in advance. Rows with coordinates containing any one of 0, k₁−1, . . . , kₙ−1 are listed. All convolution steps with a filter frame centered at these rows are skipped. The frame shift through a regular skip operation continues until the filter center reaches an endpoint (k₁−2, . . . , kₙ−2).

For example, one row is skipped when a filter center in a dimension 1 is 0 or k₁−1. k₁ rows are skipped when the filter center in dimensions 1 and 2 are (0 or k₁−1) and (0 or k₂−1), respectively. k₁×k₂ rows are skipped when the filter center in dimensions 1, 2, and 3 are (0 or k₁−1), (0 or k₂−1) and (0 or k₃−1), respectively,

FIG. 14 illustrates intermittent skipping of convolution with more-than-one-row downward shifts of the filter frame. The large columns with numbers means the convolution steps.

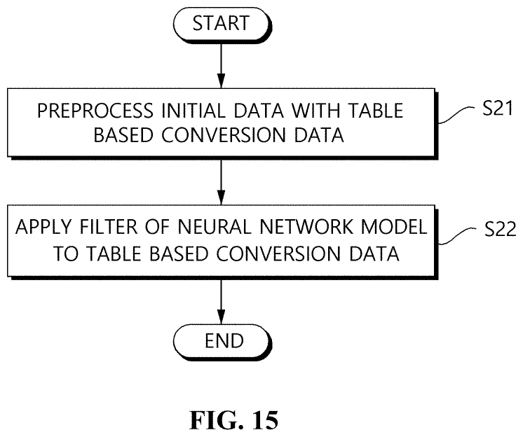

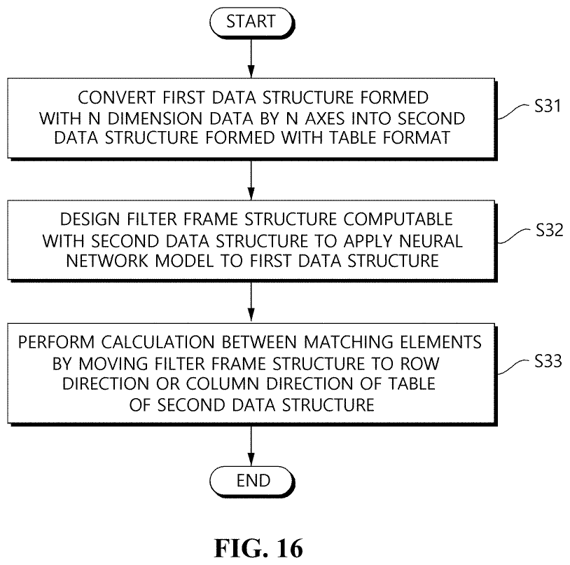

FIGS. 15 and 16 are views illustrating a data processing method according to another exemplary embodiment of the present disclosure.

The data processing method is performed by a data processing device.

The data processing method includes a step S21 of preprocessing initial data with table-based conversion data and a step S22 of applying a filter of a neural network model to the table based conversion data.

The preprocessing step S21 includes a step of converting a first data structure formed with N-dimensional data by N axes (N is a natural number of 2 or larger).

The first data structure may include a hypercube having 4D or higher dimensional depth information. The first data structure includes bio extraction data indicating a result for the flow cytometry of blood and the bio extraction data may be expressed by a predetermined standardized format or a flow cytometry standard (FCS) format.

In the second data structure, (i) coordinate information corresponding to N axes and (ii) value information matching the coordinate information are disposed with reference to a row direction or a column direction. The second data structure merges measurement values of some parameters of the bio extraction data and transforms the measurement values into data including a coordinate value for a channel and includes the transformed data and a count value.

The preprocessing step S21 includes a step S32 of designing a filter frame structure which is computable with a second data structure and expresses a dimension to apply a neural network model to the first data structure.

In the step of designing a filter frame structure, a filter center of the filter frame structure is disposed with reference to a predetermined coordinate to set a starting position of the filter frame structure.

In the step of designing a filter frame structure, filter weight elements of the filter frame structure may expand with a fractal like pattern according to a dimension with reference to the row direction or the column direction in consideration of a dimension of the first data structure.

The step S22 of applying a filter includes a step S33 of performing calculation between matching elements by moving the filter frame structure with reference to the row direction or the column direction of a table of the second data structure.

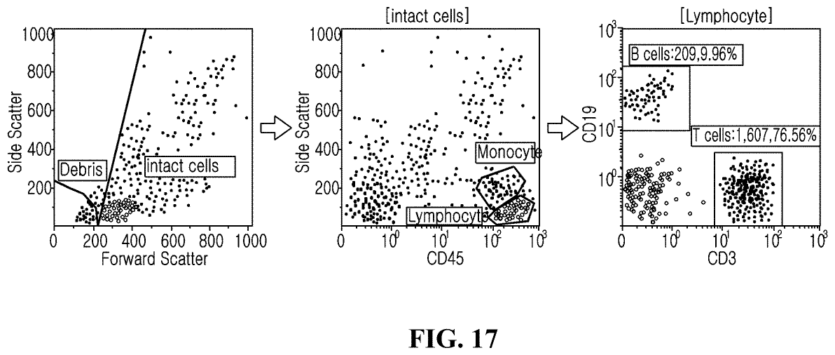

In the step S22 of applying a filter, when the filter center of the filter frame structure satisfies a predetermined row condition or column condition, the calculation may be skipped. FIG. 17 is an exemplary view for explaining an analysis operation of bio extraction data of the related art.

Generally, as illustrated in FIG. 17, the method of analyzing FCS data is configured by processes of precisely selecting/separating cells (clusters) to be analyzed based on the analyst's scientific knowledge and counting the selected cells or extracting measured optical properties (for example, light dispersion intensity or fluorescence) and related biological properties (for example, a size, a structure, or antigen phenotype).

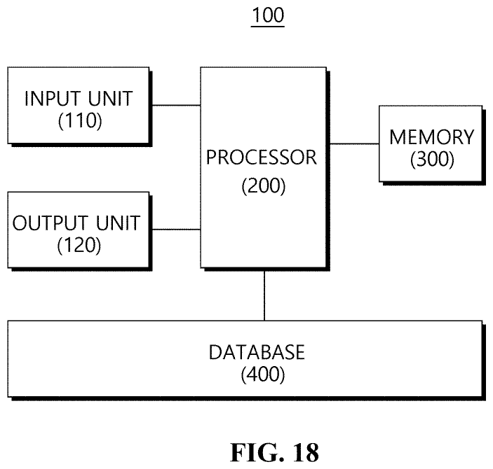

FIG. 18 is a block diagram schematically illustrating a bio extraction data based disease diagnosis device according to an exemplary embodiment of the present disclosure. The disease diagnosis device 100 according to the exemplary embodiment includes an input unit 110, an output unit 120, a processor 200, a memory 300, and a database 400. The disease diagnosis device 100 of FIG. 2 is an example so that all blocks illustrated in FIG. 18 are not essential components and in the other exemplary embodiment, some blocks included in the disease diagnosis device 100 may be added, modified, or omitted. In the meantime, components included in the disease diagnosis device 100 may be implemented by a separate software device or a separate hardware device with the software combined therewith.

The disease diagnosis device 100 performs operations of generating a predictable diagnosis model or diagnosing a specific disease by automatically preprocessing flow cytometry standard (FCS) data as learning data, utilizing the preprocessed data as data for machine learning and an artificial intelligence diagnosis model, finding features of various diseases by means of the machine learning, and identifying correlation between the features and the disease.

The input unit 110 refers to means of inputting or acquiring data for controlling the disease diagnosis device 100. The input unit 110 interworks with the processor 200 to input various types of control signals or interworks with an external device to directly acquire data to transmit the data to the processor 200.

The output unit 120 interworks with the processor 200 to display various information such as data preprocessing results, learning results, or diagnosis results. The output unit 120 may desirably display various information through a display (not illustrated) equipped in the disease diagnosis device 100, but is not necessarily limited thereto.

The processor 200 performs a function of executing at least one instruction or program included in the memory 300.

The processor 200 according to the present exemplary embodiment performs preprocessing based on bio extraction data acquired from the input unit 110 or the database 400 and performs machine learning to diagnose a disease based on the preprocessed data. Further, the processor 200 may diagnose a disease of a diagnosis target based on the trained learning result. The detailed operation of the processor 200 according to the exemplary embodiment has been described with reference to FIG. 3. Here, the bio extraction data is desirably bio extraction flow cytometry standard (FCS) raw data, but is not necessarily limited thereto.

The memory 300 includes at least one instruction or program which is executable by the processor 200. The memory 300 may include an instruction or a program for an operation of preprocessing data based on the bio extraction data.

Further, the memory 300 may include an instruction or a program for an operation of performing machine learning based on the preprocessed data. Further, the memory 300 may include an instruction or a program for an operation of diagnosing a disease of the diagnosis target based on the learning result. The database 400 refers to a general data structure implemented in a storage space (a hard disk or a memory) of a computer system using a database management program (DBMS) and means a data storage format which freely searches (extracts), deletes, edits, or adds data. The database 400 may be implemented according to the object of the exemplary embodiment of the present disclosure using a relational database management system (RDBMS) such as Oracle, Informix, Sybase, or DB2, an object oriented database management system (OODBMS) such as Gemston, Orion, or O2, and XML native database such as Excelon, Tamino, Sekaiju and has an appropriate field or elements to achieve its own function.

The database 400 according to the exemplary embodiment may store information related to the bio extraction data and provide bio extraction data and information related to the bio extraction data. The bio extraction data stored in the database 400 may be data indicating a result for flow cytometry of the blood. The bio extraction data is desirably data with a predetermined standardized format or flow cytometry standard (FCS) format data, but is not necessarily limited thereto.

It has been described that the database 140 is implemented in the disease diagnosis device 100, but is not necessarily limited thereto and may be implemented as a separate data storage device.

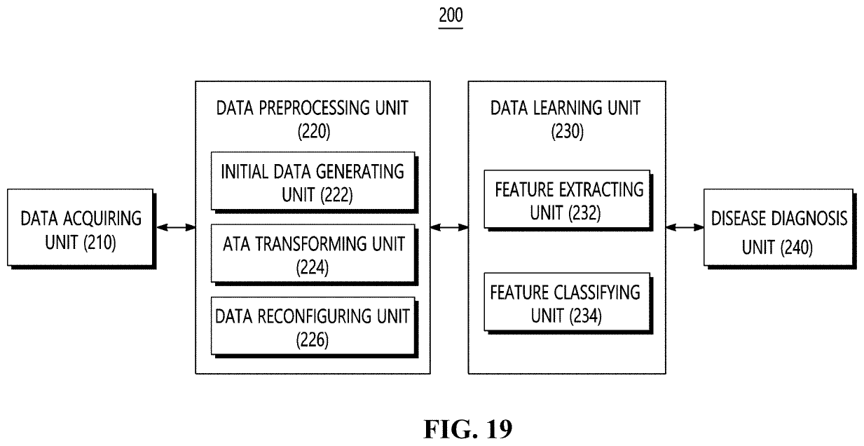

FIG. 19 is a block diagram schematically illustrating an operation configuration of a processor in a disease diagnosis device according to an exemplary embodiment of the present disclosure.

The processor 200 included in the disease diagnosis device 100 according to the exemplary embodiment includes a data acquiring unit 210, a data preprocessing unit 220, a data learning unit 230, and a disease diagnosis unit 240. The processor 200 of FIG. 19 is an example so that all blocks illustrated in FIG. 19 are not essential components and in the other exemplary embodiment, some blocks included in the processor 200 may be added, modified, or omitted. In the meantime, components included in the processor 200 may be implemented by a separate software device or a separate hardware device with the software combined therewith.

The data acquiring unit 210 performs an operation of acquiring bio extraction data extracted from the blood of the diagnosis target. Here, the bio extraction data may be data indicating a result for flow cytometry of the blood. The bio extraction data is desirably data with a predetermined standardized format or flow cytometry standard (FCS) format data, but is not necessarily limited thereto.

The data acquiring unit 210 may acquire the bio extraction data by means of the input unit 110 or the data base 400 interworking with the processor 200. Here, when the bio extraction data is acquired from the database 400 interworking with the processor 200, the data acquiring unit 210 automatically collects the bio extraction data at a predetermined cycle or collects the bio extraction data by transmitting a data request signal input through the input unit 110 to the database 400.

The data preprocessing unit 220 performs an operation of transforming the initial data generated based on a plurality of parameters into coordinate values for a plurality of channels and reconfiguring the transformed data as learning data. The data preprocessing unit 220 according to the exemplary embodiment includes an initial data generating unit 222, a data transforming unit 224, and a data reconfiguring unit 226.

The initial data generating unit 222 generates initial data using measurement values of all the plurality of parameters of a test item channel included in the bio extraction data or some parameters.

The initial data generating unit 222 generates the initial data using the measurement values of at least two of the plurality of parameters.

The data transforming unit 224 merges measurement values of all or some of parameters included in the initial data without being processed to transform the measurement values into data including coordinate values for the test item channels and generates a data table including the transformed data and count values for the transformed data.

Further, the data transforming unit 224 takes a method of transforming (image depth conversion) data by substituting a quotient obtained by dividing the measurement values of all or some parameters included in the initial data by a predetermined constant value (for example, a specific value such as 4, 8, or 32) and adding a predetermined value (for example, 10) to each quotient to prevent data loss caused at this time. A data table including the data transformed as described above and count values for the transformed data is generated.

The data transforming unit 224 transforms data into transformed data including a coordinate value generated by merging the measurement values of some parameters sequentially or in a predetermined order.

Further, when there is the same coordinate value as the coordinate value included in the transformed data, the data transforming unit 224 deletes the same coordinate value, updates a count value by increasing the count value for the coordinate value in a predetermined unit, and generates the data table including the transformed data and the updated count value.

The data reconfiguring unit 226 performs an operation of reconfiguring to a data table for machine learning using the transformed data included in the data table.

The data reconfiguring unit 226 configures the coordinate value included in the transformed data with one-dimensional coordinate value and reconfigure Πᵢ₌₁^{m} nᵢ type (ni is a natural number of a predetermined reference size value or larger) machine learning image (a data table) using a method of filling a portion which does not have a coordinate value with 0 value or displaying only a portion with a coordinate value during the process of configuring with the one-dimensional coordinate value. Here, the reconfigured machine learning image (data table) may be a two dimensional or three dimensional form.

Although it is described that the data preprocessing unit 220 according to the present exemplary embodiment is included in the disease diagnosis device 100, it is not necessarily limited thereto and the data-preprocessing unit may be implemented as a separate device from the disease diagnosis device 100. For example, the data preprocessing unit 220 may be implemented as a separate device such as a data preprocessing device (not illustrated) which converts the bio extraction data into machine learning data for diagnosis and the data preprocessing device (not illustrated) may interwork with a device which diagnoses diseases by performing the learning in various ways.

The data learning unit 230 extracts features from the reconfigured learning data and classifies the extracted features to perform the learning for disease diagnosis. The data learning unit 230 according to the present exemplary embodiment includes a feature extracting unit 232 and a feature classifying unit 234.

The feature extracting unit 232 extracts features in the reconfigured data included in the data table for machine learning using a convolution algorithm.

The feature classifying unit 234 classifies features for every specific disease to perform the learning.

The disease diagnosis unit 240 perform an operation of diagnosing a specific disease using the trained feature value. When new information for a diagnosis target is input, the disease diagnosis unit 240 compares the new information with the feature for the specific disease to diagnose the disease.

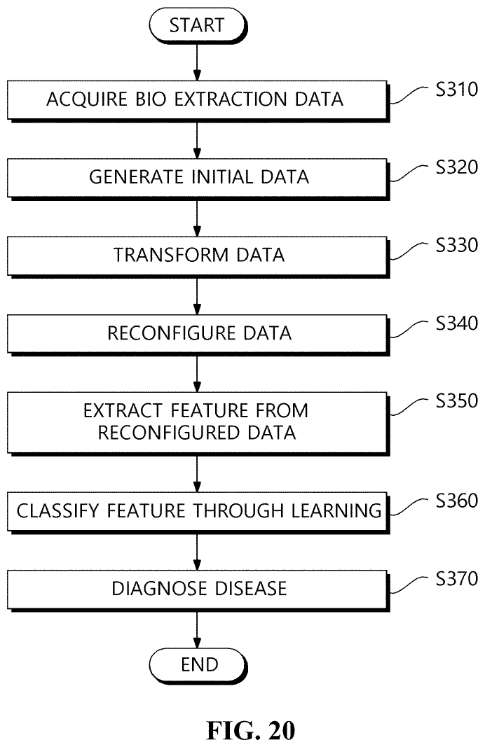

FIG. 20 is a flowchart for explaining a bio extraction data based disease diagnosis method according to an exemplary embodiment of the present disclosure.

The disease diagnosis device 100 acquires bio extraction data extracted from blood of a diagnosis target (S310). Here, the bio extraction data may be data indicating a result for flow cytometry of the blood. The bio extraction data is desirably data with a predetermined standardized format or flow cytometry standard (FCS) format data, but is not necessarily limited thereto.

The disease diagnosis device 100 generates initial data based on the bio extraction data (S320). The disease diagnosis device 100 generates initial data using measurement values of all the plurality of parameters of a test item channel included in the bio extraction data or some parameters.

The disease diagnosis device 100 transforms data included in the initial data to generate a data table (S330). The disease diagnosis device 100 merges measurement values of some of parameters included in the initial data to transform the measurement values into data including coordinate values for the test item channels and generates a data table including the transformed data and count values for the transformed data.

The disease diagnosis device 100 reconfigures transformed data included in the data table to generate a data table for machine learning (S340).

The disease diagnosis device 100 configures the coordinate value included in the transformed data included in the data table with one-dimensional coordinate value and reconfigure Πᵢ₌₁^{m} nᵢ (ni is a natural number of a predetermined reference size value or larger) machine learning image (a data table) using a method of filling a portion which does not have a coordinate value with 0 value or displaying only a portion with a coordinate value during the process of configuring with the one-dimensional coordinate value.

The disease diagnosis device 100 extracts features in the reconfigured data included in the data table for machine learning using a convolution algorithm.

The disease diagnosis device 100 performs learning based on the feature to classify the features by specific diseases (S360).

The disease diagnosis device 100 diagnoses a specific disease using the trained feature. When new information for a diagnosis target is input, the disease diagnosis device 100 compares the new information with the feature for the specific disease to diagnose the disease.

Even though in FIG. 20, it is described that the steps are sequentially performed, the present invention is not necessarily limited thereto. In other words, the steps illustrated in FIG. 20 may be changed or one or more steps may be performed in parallel so that FIG. 20 is not limited to a time-series order.

The disease diagnosis method according to the exemplary embodiment described in FIG. 20 may be implemented by an application (or a program) and may be recorded in a terminal (or computer) readable recording media. The recording medium which has the application (or program) for implementing the disease diagnosis method according to the exemplary embodiment recorded therein and is readable by the terminal device (or a computer) includes all kinds of recording devices or media in which computing system readable data is stored.

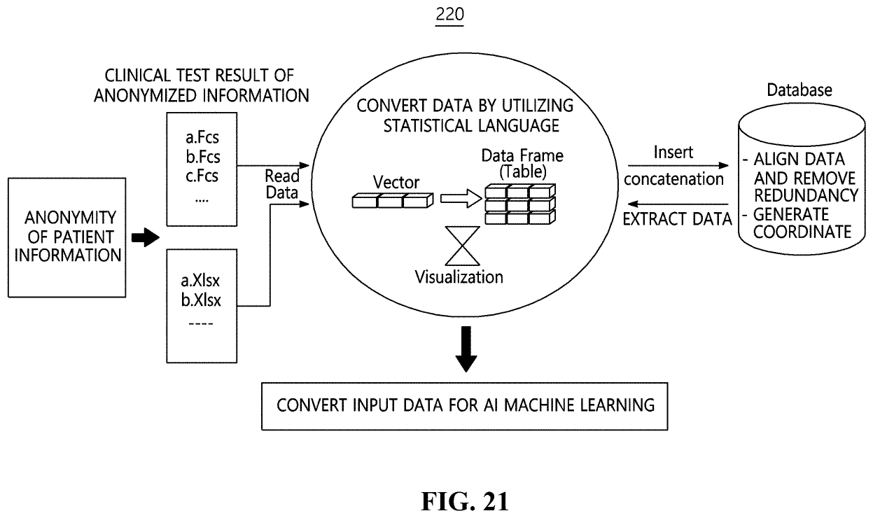

FIG. 21 is an exemplary view for explaining an operation of diagnosing a disease using patient information and bio extraction data according to an exemplary embodiment of the present disclosure. Specifically, FIG. 21 is an exemplary view for explaining a data preprocessing step of converting patient information and bio extraction FCS raw data according to an exemplary embodiment of the present disclosure into a hypercube to be applicable to a visual recognition machine learning.

The data preprocessing unit 220 in the disease diagnosis device 100 performs the data preprocessing for machine learning.

Patient information which distinguishes a diagnosis target is anonymized and a clinical test result of the anonymized information is input to the preprocessing unit.

The data preprocessing unit acquires a predetermined excel format or FCS format of bio extraction and expresses measurement values of a plurality of parameters included in the bio extraction data with a vector based coordinate value to generate initial data.

The data preprocessing unit 220 merges coordinate values of the plurality of parameters included in the initial data to be transformed into one coordinate value and generates a data table (data frame) by counting transformed data and merged coordinate values. The data preprocessing unit 220 reads or writes data stored in the database to update the data table.

The data preprocessing unit 220 reconfigures and converts the transformed data included in the data table. The data preprocessing unit 220 configures the coordinate value included in the transformed data included in the data table with one-dimensional coordinate value and reconfigure Πᵢ₌₁^{m} nᵢ type (ni is a natural number of a predetermined reference size value or larger) machine learning image (a data table) using a method of filling a portion which does not have a coordinate value with 0 value or displaying only a portion with a coordinate value during the process of configuring with the one-dimensional coordinate value.

The data preprocessing unit 220 transmits the converted machine learning data or the data table for machine learning to the data learning unit 230 to perform the learning for diagnosing a specific disease.

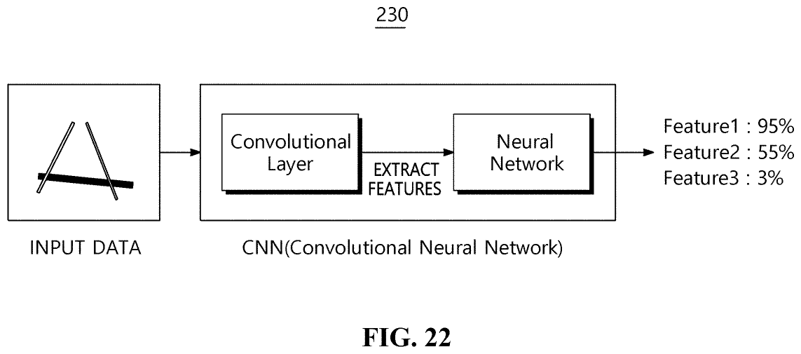

FIG. 22 is a block diagram for explaining an operation of diagnosing a disease using a neural network according to an exemplary embodiment of the present disclosure.

The data learning unit 230 performs the image learning process using machine learning data configured in the data preprocessing unit 220 as input data.

The data learning unit 230 performs an operation of detecting a feature from the input data by means of the image learning process. Here, the data learning unit 230 may detect the feature of the input data using a convolution algorithm based on a plurality of convolution layers and other advanced machine learning algorithm.

The data learning unit 230 performs the learning based on the detected features to classify features of the specific disease.

The disease diagnosis unit 240 performs the diagnosis of the disease based on the learning result of the data learning unit 230. When new data for the diagnosis target or data prior to the machine learning is input, the disease diagnosis unit 240 analyzes whether there is a feature extracted from a previously trained specific disease (for example, hematologic malignancy) patient group in the data and diagnoses the specific disease depending on the presence of the feature.

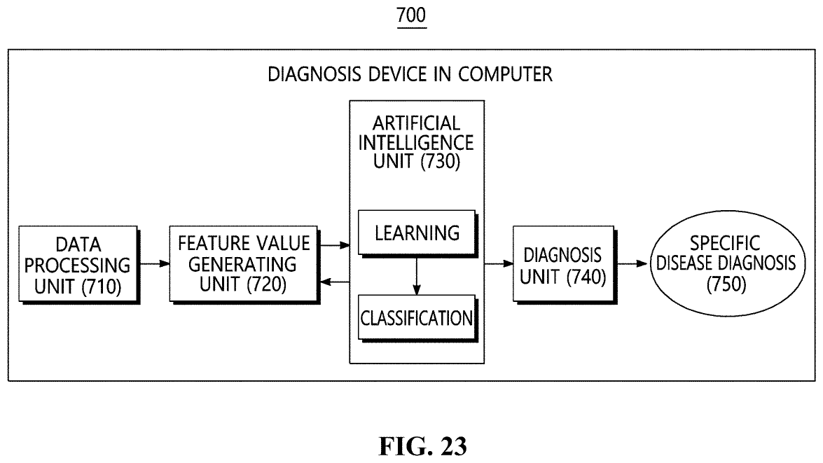

FIG. 23 is an exemplary view for explaining an operation process of a diagnosis device in a computer according to an exemplary embodiment of the present disclosure.

The disease diagnosis device 100 according to the exemplary embodiment is implemented by a diagnosis device 700 in a computer. The diagnosis device 700 in the computer may be configured to include a data processing unit 710, a feature value generating unit 720, an artificial intelligence unit 730, and a diagnosis unit 740. The data processing unit 710 performs an operation of transforming the initial data generated based on a plurality of parameters into coordinate values for a plurality of channels and reconfiguring the transformed data as machine learning data. Here, the data processing unit 710 may be implemented to include all or some of the functions of the data preprocessing unit 220.

The feature value generating unit 720 generates the feature extracted in the reconfigured data included in the data table for machine learning using a convolution algorithm or other advanced machine learning algorithm. Here, the feature generating unit 720 may be implemented to include some of the functions of the data learning unit 230.

The artificial intelligence unit 730 performs the learning based on the extracted feature and classifies the feature values for every specific disease according to the learning result. Here, the artificial intelligence unit 730 may be implemented to include some of the functions of the data learning unit 230.

The diagnosis unit 740 diagnoses a specific disease using the trained feature. When new information for a diagnosis target is input, the diagnosis unit 740 compares the new information with the feature for the specific disease to diagnose the disease. Here, the diagnosis unit 740 may be implemented to include some of the functions of the disease diagnosis unit 240.

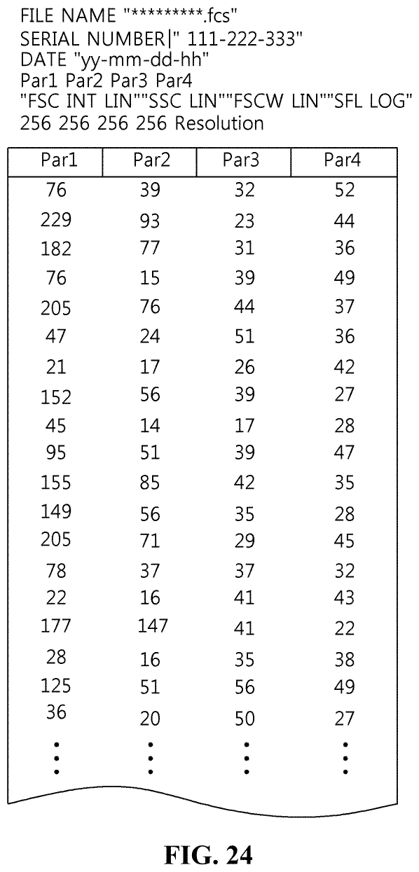

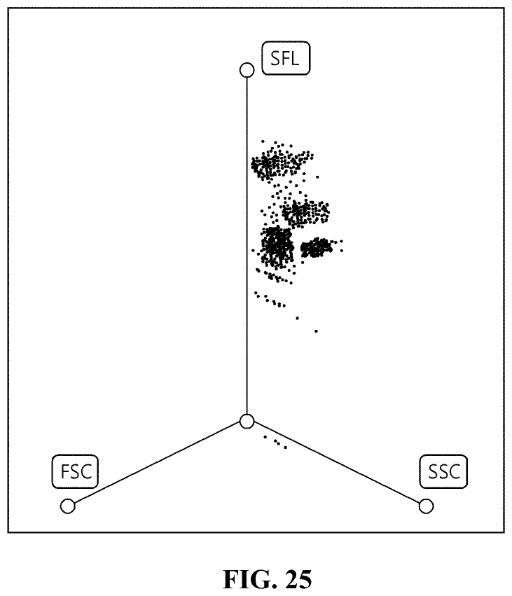

FIGS. 24 and 25 are exemplary views for explaining an operation of generating initial data based on bio extraction data according to an exemplary embodiment of the present disclosure.

Referring to FIG. 24, bio extraction data extracted from the blood of the diagnosis target includes a plurality of parameters and each of the plurality of parameters includes a measurement value. For example, the bio-extraction data extracted through an automatic blood cell analyzer is divided into two to four files for each patient, sample, and analysis module of the analysis equipment and each file may be implemented by a table format in which measurement values for every analysis parameter are listed as illustrated in FIG. 24.

For example, the bio extraction data may be a set of points formed of four-dimensional coordinates using four analysis parameters. However, for better understanding through image expression, three parameters among four parameters included in the bio extraction data are selected and three-dimensional coordinate points are expressed using the selected parameters as illustrated in FIG. 25. Here, the disease diagnosis device 100 may generate initial data for data preprocessing by means of the selected parameters.

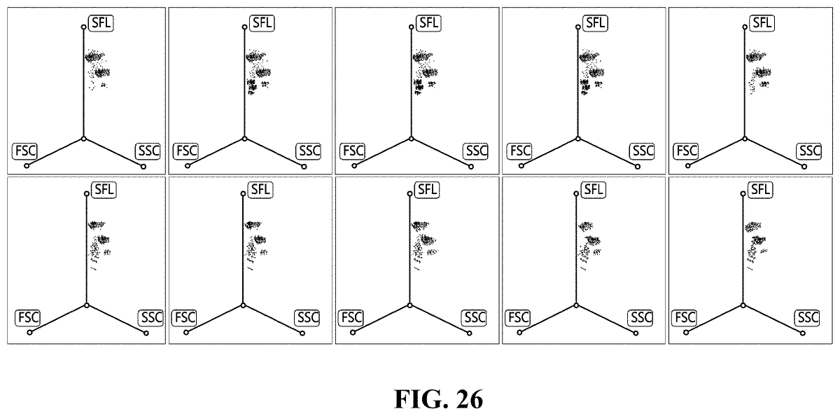

FIGS. 26 to 29 are exemplary views illustrating initial data of each of a plurality of channels according to still another exemplary embodiment of the present disclosure. FIGS. 26 to 29 are exemplary views illustrating initial data of each of the plurality of parameters (three parameters in the present example) included in the CBC based FCS data according to an exemplary embodiment of the present disclosure as a shape in a three-dimensional (hyper) cube. The shapes in 10 cubes illustrated in FIGS. 26 to 29 visualize data originating from 10 samples or 10 patients and have similar and different morphologic characteristic.

The three-dimensional coordinate points based on the bio extraction data may be graphed as a plot as illustrated in FIGS. 26 to 29. The plot pattern of the coordinate points is similar for every patient/sample, but also has subtle differences. For example, since the automatic blood analysis equipment simultaneously performs individual analysis through two to four channels (or modules), two or four FCS data for one sample may be generated.

Referring to FIGS. 26 to 29, three parameters among parameters (FCS, FCSW, SSC, SFL; four dimension) of the FCS data for every channel of the automatic blood cell analysis collected from 10 patients are listed in a three-dimensional coordinate. Ten FCS data plots for every channel were listed to enable visual comparison.

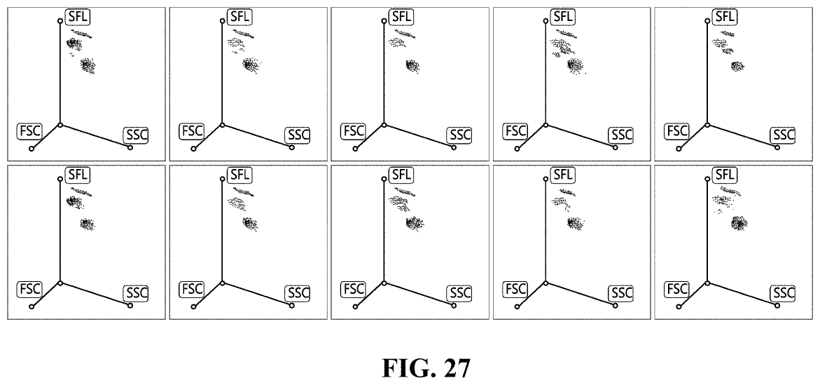

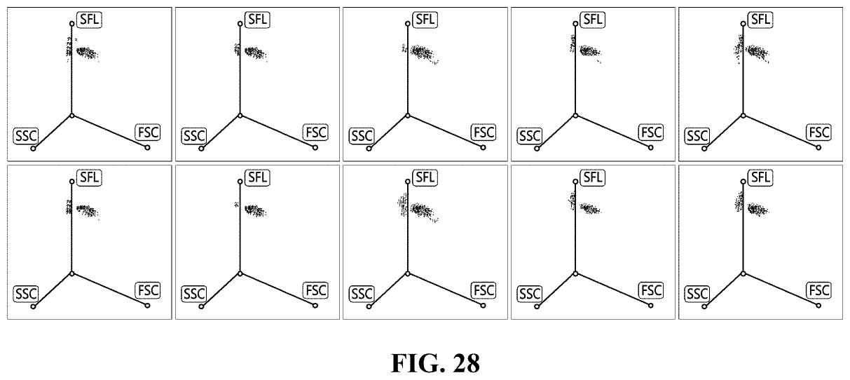

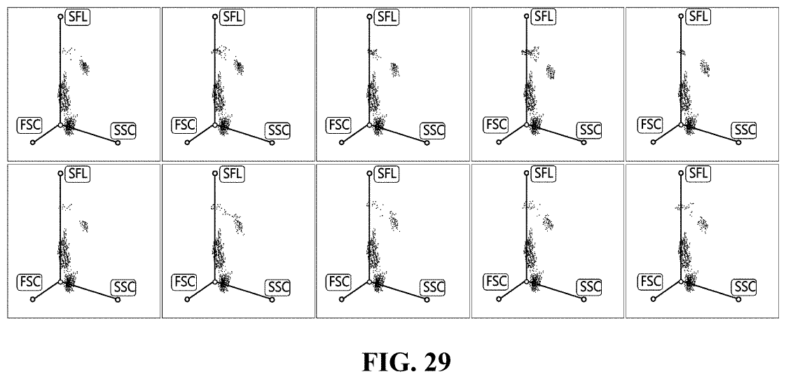

FIG. 26 is plots for a WDF channel (one of white blood analysis channels of automatic blood cell analyzer), FIG. 27 illustrates plots for a WPF channel (one of white blood analysis channels of automatic blood cell analyzer), FIG. 28 is plots for a WNR channel (a white blood analysis channel of automatic blood cell analyzer), and FIG. 29 illustrates plots for a PLT-F channel (one of blood platelet analysis channels of automatic blood cell analyzer). Each plot illustrated in FIGS. 26 to 29 shows a similar clustering pattern, but has a subtle difference in a detailed distribution pattern.

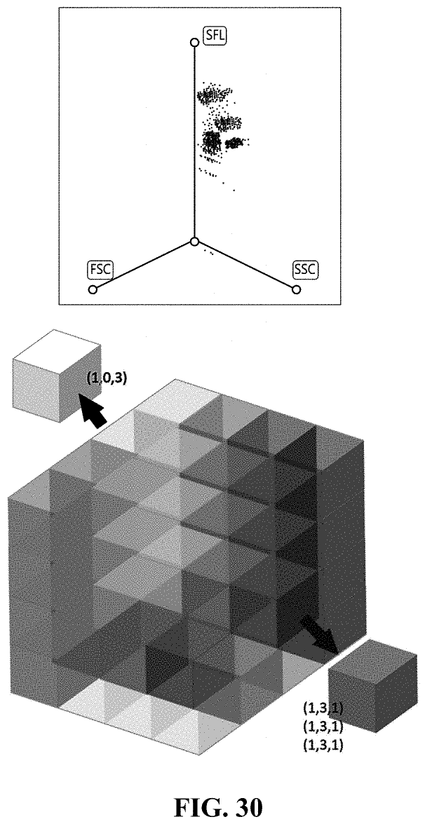

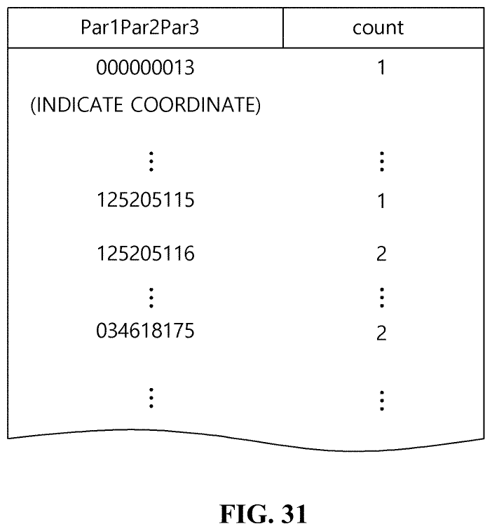

FIGS. 30 and 31 are exemplary views for explaining an operation of modifying basic data based on bio extraction data according to an exemplary embodiment of the present disclosure.

FIG. 30 is an exemplary view for explaining that the FCS data is expressed with a shape in a hypercubic space (in this example, a three-dimensional cube corresponding to three parameters). The hypercubic space is configured by a set of hypercubic pixels and a coordinate indicating a location of each pixel is a measurement value of each corresponding parameter. The gray-scale densities of each pixel is determined by a number of cells or particles having a combination of parameter values corresponding to the location of each pixel.

FIG. 31 illustrates a data table for explaining an operation of transforming initial data. FIG. 31 illustrates a relationship of a parameter value and a hypercubic pixel coordinate and a gray-scale density (count column) per pixel according to a gray-scale density definition of each pixel and explains a table listed according to a coordinate of the pixel.

The disease diagnosis device merges measurement values of parameters of the initial data (FCS data) to transform each test item value to be one coordinate value.

Further, the diagnosis device 100 takes a method of transforming (image depth conversion) data by substituting a quotient obtained by dividing the measurement values of all or some parameters included in the initial data by a predetermined constant value (for example, a specific value such as 4, 8, or 32) and adding a predetermined value (for example, 10) to each quotient to prevent data loss caused at this time.

Further, the disease diagnosis device 100 generates a data table including transformed data and count values for each transformed data.

Further, when there is the same coordinate value as the coordinate value included in the transformed data, the disease diagnosis device 100 deletes the same coordinate value, updates a count value by increasing the count value for the coordinate value in a predetermined unit, and generates the data table including the transformed data and the updated count value. For example, the disease diagnosis device 100 may generate new data table such that when there is one coordinate value of the transformed data, the disease diagnosis device 100 assigns 1 as a count value, and when there is the same coordinate value, 2 is assigned as the count value of the corresponding coordinate value.

The disease diagnosis device 100 calculates the number of coordinate points corresponding to each pixel in the coordinate space by means of the data table. In FIG. 10A, the coordinate value included in the transformed data is illustrated on a graph and FIG. 10B illustrates an operation of counting a coordinate point corresponding to each pixel in the coordinate space by means of the data table.

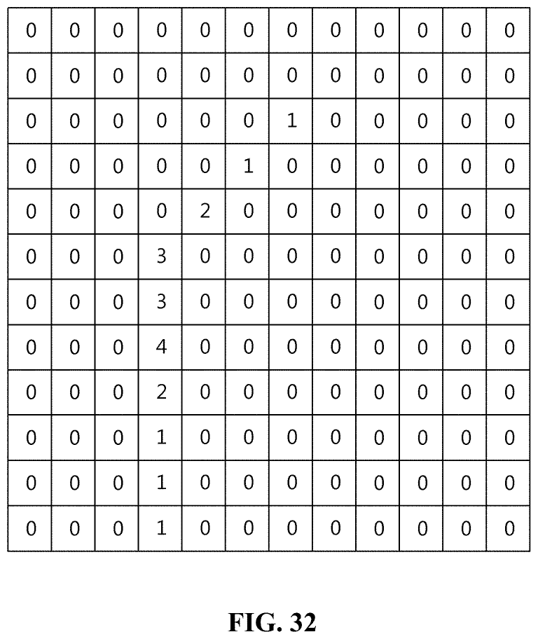

FIG. 32 is a view for explaining an operation of reconfiguring data based on bio extraction data according to an exemplary embodiment of the present disclosure. The FCS table is converted into a table representing the shape in the hypercube as in the method, and then is rearranged to be secondarily converted into a two dimensional image format.

The disease diagnosis device 100 may represent the count values displayed in the order of the coordinates of the data table as a one-dimensional arrangement of the same order, and reconfigure them into a two-dimensional array (image format) for machine learning

The disease diagnosis device 100 configures the coordinate value included in the transformed data with one-dimensional coordinate value and reconfigure Πᵢ₌₁^{m} nᵢ type (ni is a natural number of a predetermined reference size value or larger) machine learning image using a method of filling a portion which does not have a coordinate value with 0 value or displaying only a portion with a coordinate value during the process of configuring with the one-dimensional coordinate value. For example, as illustrated in FIG. 11, the disease diagnosis device 100 may reconfigure the data like a data table for machine learning with a 12×12 size. Here, one row means one coordinate value and a count value.

The device may be implemented in a logic circuit by hardware, firm ware, software, or a combination thereof or may be implemented using a general purpose or special purpose computer. The device may be implemented using hardwired device, field programmable gate array (FPGA) or application specific integrated circuit (ASIC). Further, the device may be implemented by a system on chip (SoC) including one or more processors and a controller.

The device may be mounted in a computing device or a server provided with a hardware element as a software, a hardware, or a combination thereof. The computing device or server may refer to various devices including all or some of a communication device for communicating with various devices and wired/wireless communication networks such as a communication modem, a memory which stores data for executing programs, and a microprocessor which executes programs to perform operations and instructions.

The operation according to the exemplary embodiment of the present disclosure may be implemented as a program instruction which may be executed by various computers to be recorded in a computer readable medium. The computer readable medium indicates an arbitrary medium which participates to provide an instruction to a processor for execution. The computer readable medium may include solely a program instruction, a data file, and a data structure or a combination thereof. For example, the computer readable medium may include a magnetic medium, an optical recording medium, and a memory. The computer program may be distributed on a networked computer system so that the computer readable code may be stored and executed in a distributed manner. Functional programs, codes, and code segments for implementing the present embodiment may be easily inferred by programmers in the art to which this embodiment belongs.

The present embodiments are provided to explain the technical spirit of the present embodiment and the scope of the technical spirit of the present embodiment is not limited by these embodiments. The protection scope of the present embodiments should be interpreted based on the following appended claims and it should be appreciated that all technical spirits included within a range equivalent thereto are included in the protection scope of the present embodiments.

What is claimed is:

1. A data processing method, comprising:
preprocessing initial data with table based conversion data; and
applying a filter of a neural network model to the table based conversion data.

2. The data processing method according to claim 1, wherein the preprocessing step includes:
converting a first data structure formed by N-dimensional data by N axes (N is a natural number of 2 or larger) into a second data structure formed as a table format.

3. The data processing method according to claim 2, wherein the first data structure includes a hypercube having depth information of four dimension or higher including two dimension and three dimension.

4. The data processing method according to claim 2, wherein in the second data structure, (i) coordinate information corresponding to N axes and (ii) value information matching the coordinate information are disposed with reference to a row direction or a column direction.

5. The data processing method according to claim 2, wherein the first data structure includes bio-extraction data indicating a measurement result of flow cytometry of a clinical sample of blood or a biological analysis sample and an analysis technique using flow cytometry and bio extraction data may be expressed by a predetermined standardized format or a flow cytometry standard (FCS) format, and the second data structure merges measurement values of some parameters of the bio extraction data and transforms the measurement values into data including a coordinate value for a channel and includes the transformed data and a count value.

6. The data processing method according to claim 2, wherein the preprocessing step includes:
designing a filter frame structure which is computable with a second data structure and expresses a dimension to apply a neural network model to the first data structure.

7. The data processing method according to claim 6, wherein in the designing of a filter frame structure, a filter center of the filter frame structure is disposed with reference to a predetermined coordinate to set a starting position of the filter frame structure.

8. The data processing method according to claim 7, wherein in the designing of a filter frame structure, filter weight elements of the filter frame structure expands with a fractal like pattern according to a dimension with reference to the row direction or the column direction in consideration of a dimension of the first data structure.

9. The data processing method according to claim 6, wherein in the applying of a filter, the calculation is performed between matching elements by moving the filter frame structure with reference to the row direction or the column direction of a table of the second data structure.

10. The data processing method according to claim 9, wherein in the applying of a filter, when the filter center of the filter frame structure satisfies a predetermined row condition or column condition, the calculation is skipped.

11. A data processing device including a processor, wherein the processor preprocesses initial data with table based conversion data and applies a filter of a neural network model to the table based conversion data.

12. The data processing device according to claim 11, wherein the processor converts a first data structure formed by N-dimensional data by N axes (N is a natural number of 2 or larger) into a second data structure formed as a table format.

13. The data processing device according to claim 12, wherein the first data structure includes a hypercube having depth information of four dimension or higher including two dimension and three dimension.

14. The data processing device according to claim 12, wherein in the second data structure, (i) coordinate information corresponding to N axes and (ii) value information matching the coordinate information are disposed with reference to a row direction or a column direction.

15. The data processing device according to claim 12, wherein the first data structure includes bio-extraction data indicating a measurement result of flow cytometry of a clinical sample of blood or a biological analysis sample and an analysis technique using flow cytometry and bio extraction data is expressed by a predetermined standardized format or a flow cytometry standard (FCS) format, and the second data structure merges measurement values of some parameters of the bio extraction data and transforms the measurement values into data including a coordinate value for a channel and includes the transformed data and a count value.

16. The data processing device according to claim 12, wherein the processor designs a filter frame structure which is computable with a second data structure and expresses a dimension to apply a neural network model to the first data structure.

17. The data processing device according to claim 16, wherein the processor disposes a filter center of the filter frame structure with reference to a predetermined coordinate to set a starting position of the filter frame structure.

18. The data processing device according to claim 16, wherein the processor expands filter weight elements of the filter frame structure with a fractal like pattern according to a dimension with reference to the row direction or the column direction in consideration of a dimension of the first data structure.

19. The data processing device according to claim 16, wherein the processor performs the calculation between matching elements by moving the filter frame structure with reference to the row direction or the column direction of a table of the second data structure.

20. The data processing device according to claim 19, wherein when the filter center of the filter frame structure satisfies a predetermined row condition or column condition, the processor skips the calculation.
